(19) United States
(12) Patent Application Publication
(10) Pub. No.: US 2013/0273501 A1
(43) Pub. Date: Oct. 17, 2013

(54) METHOD OF MAKING DENTAL PROSTHESIS AND DUCTILE ALLOYS FOR USE THEREIN

(71) Applicant: THE ARGEN CORPORATION, San Diego, CA (US)

(72) Inventors: Paul J. Cascone, Del Mar, CA (US); Arun Prasad, Cheshire, CT (US)

(73) Assignee: The Argen Corporation, San Diego, CA (US)

(21) Appl. No.: 13/861,222

(22) Filed: Apr. 11, 2013

Related U.S. Application Data
(60) Provisional application No. 61/623,595, filed on Apr. 13, 2012.

(51) Int. Cl. A61C 13/083; A61K 6/02; A61C 13/08; A61K 6/04

(52) U.S. Cl. CPC A61C 13/0835; A61K 6/04; A61K 6/0205; A61C 13/081
USPC 433/207; 29/896.1

(57) ABSTRACT

A dental prosthesis comprising a metal alloy pre-form and a dental porcelain veneer coating the metal alloy, wherein the metal alloy has a composition comprising, in % by weight, about 30-40% Co, 25-40% Ru, 20-40% Cr, and 0-0.1% Ni and wherein a coefficient of thermal expansion of the metal alloy is compatible with that of the dental porcelain to prevent cracking of the porcelain

### BACKGROUND OF THE INVENTION

1. Field of the Invention

The present invention relates generally to the manufacture of dental prostheses and, more specifically, to ductile cobalt-ruthenium-chromium alloys for use in the process.

2. Description of the Related Art

The traditional materials used for the fabrication of dental prosthetic devices have been gold and palladium based alloys. Over the last fifteen years the increasing prices of gold and palladium has prompted a search for lower cost substitute materials. Such alloys are described by Cascone in U.S. Pat. No. 7,794,652. By employing ruthenium in the alloy composition, the intrinsic cost of the alloys is lowered while still maintaining the ADA Classification of Noble (alloys that contain at least 25 weight percent gold and/or platinum group elements).

At the same time, the dental laboratory industry has been exploring more efficient methodologies in fabricating dental prosthesis. One technology that holds particular promise is selective laser melting. This process is described in http://en.wikipedia.org/wiki/Selective laser melting.

An excerpt from the reference:

Selective laser melting (SLM) is an additive manufacturing process that uses 3D CAD data as a digital information source and energy in the form of a high powered laser beam (usually an ytterbium fiber laser) to create three-dimensional metal parts by fusing fine metallic powders together. The process starts by slicing the 3D CAD file data into layers, usually from 20 to 100 micrometres thick, creating a 2D image of each layer; this file format is the industry standard .stl file used on most layer-based 3D printing or stereolithography technologies. This file is then loaded into a file preparation software package that assigns parameters, values and physical supports that allow the file to be interpreted and built by different types of additive manufacturing machines.

With SLM, thin layers of atomized fine metal powder are evenly distributed using a coating mechanism onto a substrate plate, usually metal, that is fastened to an indexing table that moves in the vertical (Z) axis. This takes place inside a chamber containing a tightly controlled atmosphere of inert gas, either argon or nitrogen at oxygen levels below 500 parts per million. Once each layer has been distributed, each 2D slice of the part geometry is fused by selectively applying the laser energy to the powder surface, by directing the focused laser beam using two high frequency scanning mirrors in the X and Y axes. The laser energy is intense enough to permit full melting (welding) of the particles to form solid metal. The process is repeated layer after layer until the part is complete.

It is known to manufacture dental prostheses using direct metal laser sintering by fusing cobalt chromium alloy powder using a laser, see: www.eos.info/en/applications/.

The SLM process requires the use of fine alloy power. When alloys described by Cascone in U.S. Pat. No. 7,794,652 are atomized and used in the SLM machine, the parts fracture. The cause of the fractures is suspected to be large thermal stresses that arise during the rapid melting by the laser and solidification of the alloy.

### SUMMARY OF THE INVENTION

Briefly stated, the present invention is directed to a method for making a dental prosthesis comprising the steps of (a) providing a pre-alloyed fine powder comprising in % by weight: 30-40% Co, 25-40% Ru, and 20-40% Cr; and (b) forming the dental prosthesis by selective laser melting the pre-alloyed powder in a mold of a selected shape. The dental prosthesis metal alloy pre-form is then surface coated (veneering) with dental porcelain. The coefficient of thermal expansion of the metal alloy closely matches that of the dental porcelain so as to prevent cracking during high temperature processing of the porcelain. Alternatively, the method comprises the steps of (a) providing a molten bath of a ductile alloy comprising in % by weight: 30-40% Co, 25-40% Ru, and 20-40% Cr; casting the molten alloy into a mold to form a near-net shape pre-form or blank of a dental prosthesis; machining, as by grinding, the pre-form or blank to a selected shape; and coating the machined shape with a dental porcelain.

### BRIEF DESCRIPTION OF THE DRAWINGS

FIG. 1 is a photograph of a dental coping or pre-form of the present invention; and

FIG. 2 is a photograph of a finished dental prosthesis or crown of the invention after a porcelain coating is applied to the coping of FIG. 1.

### DETAILED DESCRIPTION OF THE INVENTION

One approach to address the absorption of the stresses is to utilize ductile alloys. Such ductile alloys are described in prior art by German Patent No. 1104195 to Obrowski. We have determined the ranges described by Obrowski (29-45 wt % cobalt, 20-50 wt % ruthenium, and 20-40 wt % chromium) are broad enough to accommodate altering the thermal expansion of the alloy in order to be compatible with different dental porcelains. It is important that the coefficients of thermal expansion of the alloy and the dental porcelain closely match to prevent cracking of the porcelain during processing which takes place at about 900° C. Currently, dental porcelains have a coefficient of thermal expansion of between about 12-14 (10⁻⁶/K at 600° C.).

The alloy used in the present invention for the manufacture of dental prosthesis is preferably in % by weight: 30-40% Co, 25-40% Ru, 20-40% Cr, and 0-0.1% Ni. The alloy contains a minimum of 25% Ru and platinum group elements, including Pt, Rh, Os, Pd, Ir, and Ru. The alloy is most preferably free of Ni and the finished part exhibits weak ferromagnetic properties, that is, the part is only weakly attracted to a magnet.

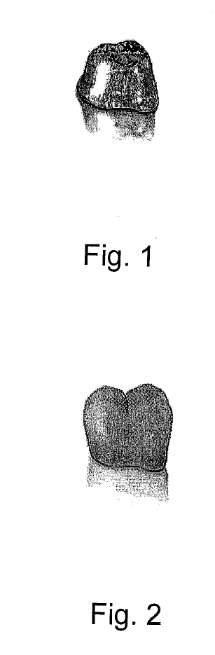

A first sample of the alloy of the invention was prepared by melting 35 wt % Co, 35 wt % Ru, and 30 wt % Cr in an induction heated crucible in an argon atmosphere. The molten alloy was then atomized to form a pre-alloyed powder. The powder was sized by screening—45 μm+10 μm. The screened fine, pre-alloyed powder was then introduced to a selective laser melting (SLM) machine to laser melt the powder and fill a mold which has been previously made to the desired shape of the dental coping (one tooth) or bridge (more than one tooth). The finished coping is shown in FIG. 1 wherein the SLM alloy is in a solidified condition. The first sample of the alloy after melting and solidification had a coefficient of thermal expansion of about 13 (10⁻⁶/K at 600° C.). The coping of FIG. 1 is then sent to a dental lab for coating (veneering) with a dental porcelain as shown in FIG. 2 as a finished dental prosthesis in the form of a crown.

It should be mentioned that the creation of a mold for a dental coping is, in itself, well known in the art and need not be explained in detail herein.

In a further embodiment of the present invention, the molten alloy of the above-described composition may be cast into a mold to form a near net shape of the coping or cast as a block or blank of metal alloy. The solidified shape or blank is then machined by grinding, for example, into the finalized prosthesis shape.

As mentioned above, the alloy of the invention preferably contains less than 0.1 wt % Ni and, more preferably, contains no Ni. The finished part does not exhibit strong ferromagnetic properties which is desirable in a dental prosthesis.

A second sample of the alloy of the present invention was prepared, having a composition containing: 40 wt % Co, 30 wt % Ru, and 30 wt % Cr. The second sample was processed in the same manner as described above with respect to the first sample alloy, including the selective laser melting. The solidified metal alloy of the second sample had a coefficient of thermal expansion of about 12 (10⁻⁶/K at 600° C.). The second sample thus provided a substantially perfect match with a dental porcelain veneer having a coefficient of thermal expansion of 12 (10⁻⁶/K at 600° C.). As mentioned above, this close match in the thermal expansion of the metal alloy and the porcelain veneer provides a crack-free dental prosthesis.

While specific embodiments of the invention have been described in detail, it will be appreciated by those skilled in the art that various modifications and alternatives to those details could be developed in light of the overall teachings of the disclosure. The presently preferred embodiments described herein are meant to be illustrative only and not limiting as to the scope of the invention which is to be given the full breadth of the appended claims and any and all equivalents thereof.

What is claimed is:

1. A dental prosthesis comprising a metal alloy pre-form and a dental porcelain veneer coating the metal alloy, wherein the metal alloy has a composition comprising, in % by weight, about 30-40% Co, 25-40% Ru, 20-40% Cr, and 0-0.1% Ni and wherein a coefficient of thermal expansion of the metal alloy is compatible with that of the dental porcelain to prevent cracking of the porcelain.

2. The dental prosthesis of claim 1, wherein the metal alloy nominally contains about 35% Co, about 35% Ru, and about 30% Cr.

3. The dental prosthesis of claim 1, wherein the metal alloy nominally contains about 40% Co, about 30% Ru, and about 30% Cr and has a coefficient of thermal expansion of about 12 (10⁻⁶/K at 600° C.).

4. A method of making a dental prosthesis comprising the steps of:
(a) providing a metal alloy according to any of claim 1, 2, or 3;
(b) providing a mold of a selected shape of a dental prosthesis;
(c) melting the alloy of step (a) and filling the mold of step (b) with the molten alloy;
(d) solidifying the alloy in the mold to provide a metal alloy pre-form; and
(e) applying a veneer of a dental porcelain to the metal alloy pre-form.

5. The method of claim 4 wherein the melting and filling of step (c) includes the use of selective laser melting or casting.
